(19) United States
(12) Patent Application Publication
(10) Pub. No.: US 2004/0138291 A1
(43) Pub. Date: Jul. 15, 2004

(54) Treatment of sexual dysfunction

(76) Inventors: Michael A. Adams, Kingston (CA); Jeremy P.W. Heaton, Gananoque (CA); Taben M. Hale, Montreal (CA); Rochard K.S. Beharry, Markham (CA)

Correspondence Address: Stephen J. Scribner, PARTEQ Innovations, Room 1625, Biosciences Complex, Queen's University, Kingston, ON, K7L 3N6, CA

(21) Appl. No.: 10/682,363

(22) Filed: Oct. 10, 2003

Related U.S. Application Data
(60) Provisional application No. 60/417,166, filed on Oct. 10, 2002.

(51) Int. Cl. A61K 31/401

(52) U.S. Cl. 514/423

(57) ABSTRACT

This invention relates to a method of normalizing sexual response in a subject, comprising administering to a subject in need thereof an anti-hypertensive therapy and a centrally-acting dopaminergic agonist. The subject may be a human male or female. In a preferred embodiment, the anti-hypertensive therapy is an ACE inhibitor such as enalapril, and the centrally-acting dopaminergic agonist is apomorphine or a pharmaceutically acceptable salt or ester thereof.

### RELATED APPLICATIONS

This application claims the benefit of the filing date of U.S. Provisional Patent Application No. 60/417,166, filed Oct. 10, 2002, the contents of which are incorporated herein by reference in their entirety.

### FIELD OF THE INVENTION

This invention relates generally to the treatment of sexual dysfunction. In particular, this invention relates to methods for treating sexual dysfunction using anti-hypertensive therapy and a centrally acting dopaminergic agonist of sexual response.

### BACKGROUND OF THE INVENTION

Recent studies suggest that at least 43%⁽²⁾ of women have some form of sexual dysfunction, which can be categorized into four main areas: 1) sexual desire disorders, namely hypoactive sexual desire or sexual aversion disorder; 2) sexual arousal disorders; 3) orgasmic disorders; and 4) sexual pain disorders which include dyspaureunia and vaginismus⁽¹⁾. The dominant category of female sexual dysfunction (FSD) is female sexual arousal disorder (FSAD), which affects up to 75% of women diagnosed with FSD.

Female sexual arousal consists of a series of vasocongestive and lubricative events, resulting primarily in increased blood flow to clitoral, labial, and vaginal tissue, and vaginal lubrication^{(3,4)}. The clitoris plays a major role during sexual activity in that it contributes to local autonomic and somatic changes causing vaginal vasocongestion, engorgement, and subsequent effects, lubricating the introital canal making sexual intercourse easier, more comfortable, and more pleasurable. During sexual arousal, blood flow to the corpora cavernosa of the clitoris cause their enlargement and tumescence.

Vaginal wall engorgement enables a process of plasma transduction to occur, allowing a flow through the epithelium and onto the vaginal surface. Plasma transduction results from the rising pressure in the vaginal capillary bed during sexual arousal. In addition, there is an increase in vaginal length and lumenal diameter, especially in the distal ⅔ of the vaginal canal.

The generation of penile and clitoral erections, vaginal and labial engorgement, and vaginal lubrication are dependent on adequate blood flow to vascular beds which feed these organs. Both smooth muscle relaxation of the corpora cavernosa as well as vasodilation of genital arterial vessels mediate the physiological response. One of the etiologies of erectile dysfunction is, thus, inadequate genital arterial inflow. If there is an inappropriate narrowing in the supporting vasculature that is not associated with an increase in perfusion pressure, blood flow into the organs at maximum dilation may be reduced and thus insufficient for the generation of an erection. There is increasing recognition that erectile dysfunction, although associated with, may appear prior to the onset of clinical signs of cardiovascular disease and therefore may be an early harbinger of progressing changes.

Thus, women with FSAD suffer from decreased blood flow to the clitorus, labia, and vaginal walls, and decreased vaginal lubrication. This is often accompanied by, or results in, other sexual dysfunctions such as dyspareunia.

Peripheral innervation of the female genital tract occurs via central-peripheral connections which are anatomically similar to those found in the male⁽⁵⁾. At the level of the female genitalia the sympathetic, parasympathetic and non-adrenergic non-cholinergic (NANC) systems are responsible for the primary neural regulation of blood flow. The precise contribution (facilitory/inhibitory) of the cholinergic (parasympathetic) and adrenergic (sympathetic) systems have not been elucidated. However, NANC factors such as nitric oxide (NO), vasoactive intestinal peptide (VIP), and calcitonin gene related peptide (cGRP) have all been implicated as factors responsible for increasing blood flow to the genital areas^{(4,7,8)}.

The etiology of sexual dysfunction may include vascular/endothelial disease such as hypertension^{(6,8,9)}, neurological disorders⁽³⁾, and hormonal disorders, such as decreased levels of estrogen and/or testosterone. FSD can also be caused, or exacerbated, by medication such as antidepressants. In particular, an understanding of the hormonal variations throughout the reproductive cycle in females must be factored in when developing possible therapeutics. Clearly FSD is a complex disorder with more active clinical issues than the corresponding male disorder, penile erectile dysfunction (MED). Hence it is not surprising that to date there has been little success in treating FSD, and use of treatments that are successful in treating MED, such as sildenafil, have shown only limited success in ameliorating FSD⁽¹⁰⁾. It may be that a different spectrum of activities, mechanisms, dosing regimens and duration of action of agents is needed when devising treatments for women, relative to approaches taken with men.

### SUMMARY OF THE INVENTION

According to one aspect of the invention there is provided a method of normalizing sexual response in a subject, comprising administering to a subject in need thereof an anti-hypertensive therapy and a centrally-acting dopaminergic agonist, such that sexual response is normalized. Preferably, subject is a human male or female. In various embodiments, the anti-hypertensive therapy is selected from the group consisting of ACE inhibitors, angiotensin II receptor antagonists, calcium channel blockers, α₁-adrenergic receptor antagonists, β-adrenergic receptor antagonists, and endothelin receptor antagonists, and the centrally-acting dopaminergic antagonist is apomorphine or a pharmaceutically acceptable salt or ester thereof.

According to another aspect of the invention there is provided a method of ameliorating sexual dysfunction in a subject comprising administering to a subject in need thereof an anti-hypertensive therapy and a centrally-acting dopaminergic agonist, such that sexual dysfunction is ameliorated. Preferably, the subject is human, and may be male or female. In one embodiment, the sexual dysfunction is female sexual arousal disorder. In another embodiment, the sexual dysfunction is male erectile dysfunction.

According to another aspect of the invention there is provided a method of enhancing the efficacy of a centrally-acting dopaminergic receptor agonist in the treatment of sexual dysfunction, comprising administering to a subject in need thereof an anti-hypertensive therapy and centrally-acting dopaminergic receptor agonist. In one embodiment, said anti-hypertensive therapy comprises administration of an ACE inhibitor over a period of time ranging between about five days to about 21 days at a dose ranging between about 1 mg to about 300 mg per kg of body weight per day. In a preferred embodiment, said ACE inhibitor is enalapril, and said enalapril is administered at a dose of about 30 mg per kg of body weight per day.

In a further embodiment, said dopaminergic receptor agonist is apomorphine or a pharmaceutically acceptable salt or ester thereof, administered on an as-needed basis. In various embodiments the apomorphine is administered in an amount between about 25 μg/kg of body weight and about 80 μg/kg of body weight, or to maintain a plasma concentration of apomorphine at a level no more than about 5.5 ng/ml. In various embodiments, apomorphine is administered via a route selected from subcutaneous, intramuscular, transdermal, sublingual, buccal, intranasal, vaginal, and rectal.

In one embodiment, the anti-hypertensive therapy and the centrally-acting dopaminergic agonist are administered at the same time. In another embodiment, the anti-hypertensive therapy and the centrally-acting dopaminergic agonist are administered chronically. In another embodiment, the anti-hypertensive therapy and the centrally-acting dopaminergic antagonist are administered together as a single dosage unit. In a preferred embodiment, the anti-hypertensive therapy is an ACE inhibitor and the centrally-acting dopaminergic antagonist is apomorphine or a pharmaceutically acceptable salt or ester thereof.

According to another aspect of the invention there is provided a pharmaceutical composition comprising a single dosage unit of an anti-hypertensive therapy and a centrally-acting dopaminergic-antagonist together with a pharmaceutically acceptable carrier therefore. In various embodiments, the anti-hypertensive therapy is selected from the group consisting of ACE inhibitors, angiotensin II receptor antagonists, calcium channel blockers, α₁-adrenergic receptor antagonists, β-adrenergic receptor antagonists, and endothelin receptor antagonists, and the centrally-acting dopaminergic antagonist is apomorphine or a pharmaceutically acceptable salt or ester thereof. In one embodiment, the anti-hypertensive therapy is an ACE inhibitor. In a preferred embodiment, the ACE inhibitor is enalapril.

According to another aspect of the invention there is provided a kit comprising a dosage unit of an anti-hypertensive therapy and a dosage unit of a centrally-acting dopaminergic antagonist, each together with a pharmaceutically acceptable carrier therefore. In one embodiment, the anti-hypertensive therapy is administered chronically and the centrally-acting dopaminergic antagonist is administered on an as-needed basis. In a preferred embodiment, the anti-hypertensive therapy is an ACE inhibitor such as enalapril and the centrally-acting dopaminergic antagonist is apomorphine or pharmaceutically acceptable salt or ester thereof.

### BRIEF DESCRIPTION OF THE DRAWINGS

Embodiments of the invention will now be described, by way of example, with reference to the accompanying drawings, wherein:

FIG. 1 is a plot showing the average number of APO-induced genital vasocongestive arousal (GVA) responses of aged female rats prior to anti-hypertensive treatment (Pre), during the two weeks of anti-hypertensive treatment (Wk1, Wk2), and during weeks 1, 3, 5, and 10 after anti-hypertensive treatment was stopped (Off1, Off3, Off5, and Off10);

FIG. 2 is a plot showing an increase in APO-induced GVA responses of aged female rats during the second week (Wk2) of anti-hypertensive treatment and up to 10 weeks after anti-hypertensive treatment was stopped (Off1 to Off10), and also an increase in the number of partial behavioural responses (corresponding stippled bars) that persisted after withdrawal of the treatment;

FIG. 3 is a plot showing age-related decrease in apomorphine-induced penile erectile responses in 30 to 68 week-old spontaneously hypertensive rats (SHR). Asterisks indicate significantly different from 30 weeks, and crosses indicate significantly different from 35 weeks;

FIG. 4 is a plot showing effect of brief, moderate anti-hypertensive therapy on penile erectile response in 30 to 32 week-old SHR;

FIG. 5 is a plot showing effect of a second anti-hypertensive treatment on penile erectile response in approximately-49 week-old SHR; and

FIGS. 6A and 6B are plots showing effect of anti-hypertensive treatment on renile erectile response at about 68 weeks of age in previously untreated SHR (A) and in previously treated SHR (B). The first bar of each triplet was at 65 weeks and the middle bar at 68 weeks, 1 week after testosterone administration.

### DETAILED DESCRIPTION OF THE INVENTION

Previous attempts to develop animal models of female sexual response have, for the most part, been limited to behavioral paradigms. A common approach in previous studies has been the assessment of female sexual receptivity based on a surrogate marker, lordotic posturing (in which the female rat demonstrates concave back flexion, lateral tail deviation and neck extension⁽¹¹⁾ in relation to male copulatory contact^{(11,12,13)}). A problem with this approach is that these behavioral responses persist despite lesioning of the hypothalamus⁽¹⁴⁾, a key central site for initiation of neurogenic sexual signalling^{(15,16)}. Furthermore, such female behavioral responses do not necessarily include the peripheral manifestation of increased blood flow to the genitalia during sexual arousal. Thus, female sexual dysfunction, originating at the level of the pudendal vasculature, may not be detected using such behavioral paradigms alone.

In males, penile erection is the standard response for assessing sexual arousal. In this regard, the well-established bio-assay of apomorphine (APO)-induced erectile function in the rat⁽¹⁷⁾ provides an animal model for this phase of the male sexual response. Apomorphine is a centrally-acting sexual response agonist which elicits erections by acting on the dopamine receptors of the midbrain, e.g., type 2 (D2) receptors of the paraventricular nucleus (PVN). This APO-induced response, in the male rat, involves both a patterned behavioral response and a physiological (vascular) change in the genitalia⁽¹⁸⁾.

As described in detail in Example 1, below, apomorphine induces a spontaneous sexual arousal response in female rats. This response includes a behavioural pattern (see Example 1), and the critical endpoint is an APO-induced genital vasocongestive arousal (GVA) response. That is, following APO administration there is a distinct, visible engorgement of the external genitalia. Moreover, this response is dependent on hormonal milieu, as ovariectomy markedly attenuates the APO-induced GVA response. The APO-induced GVA response in female rats described herein thus provides a convenient bio-assay for the study of female sexual dysfunction, such as FSAD.

We have recently demonstrated, using the well-established APO-induced erection model of male sexual response, that penile erectile dysfunction exhibited by aged spontaneously hypertensive rats (SHR) can be successfully treated with a brief anti-hypertensive therapy, resulting in a persistent reduction of the ‘off’-treatment level of arterial blood pressure and a recovery of erectile function⁽²³⁾. Further, the treatment was also found to induce regression of vascular structure and decreased responsiveness to α₁-adrenoceptor signaling. Both of these changes could be causally-linked with the improved erectile function.

Using the above-described bio-assay for the study of female sexual dysfunction, the present inventors found an age-related decrease in APO-induced GVA responses in aged (18 months) female Wistar rats. Subsequently, it was demonstrated that brief anti-hypertensive therapy restored APO-induced sexual response in aged female rats. The data indicate that both sexual behaviour and peripheral genital function are enhanced by administration of therapies that treat hypertension and modulate central sexual pathways. The invention is thus based, at least in part, on the surprising discovery that anti-hypertensive therapy restores central and peripheral sexual response.

In accordance with a broad aspect of the invention there is provided a method of normalizing sexual response in a mammal, comprising administering to a mammal in need thereof an anti-hypertensive therapy and a centrally-acting dopaminergic agonist, such that sexual response is normalized. Preferably, the mammal is a human male or female.

As used herein, the term “administering” encompasses administering one or more agents. When two or more agents are administered, they can be administered at the same time (i.e., “co-administered”), or at different times. Such co-administration includes the administration of two or more agents, such as an anti-hypertensive therapy and a centrally-acting dopaminergic agonist, at the same time, wherein the two or more agents may be in separate dosage units or the two or more agents may be combined in a single dosage unit.

As used herein, the term “therapy” relates to the dosing regimen of an agent. For example, a therapy can be chronic, in which a therapeutic agent is administered at substantially regular intervals for a prolonged period. A therapy can also be brief, in which a therapeutic agent is administered for a prescribed period, stopped for a prescribed period, and optionally administered again for a prescribed period, and so on. Thus, a therapy may include the persistent result of a previous administration. Further, a therapy can be administration of a therapeutic agent on an as-needed basis.

As used herein, the term “anti-hypertensive therapy” relates to the administration of an anti-hypertensive agent. The term “anti-hypertensive agent” as used herein denotes a therapeutic agent which acts either directly or indirectly to normalize circulatory responses. Thus, although such-agents are generally recognized for their-ability to lower blood pressure, it will be understood that these agents also have other actions, including, for example, decreasing vasoconstrictor tone, increasing endothelial function, enhancing flow mediated dilatation, enhancing delivery of other therapeutic agents to regions and tissues of the body, increasing nerve-mediated vasodilation, and improving oxygenation to various tissues. The invention relates to the use of such agents in both hypertensive and normotensive patients. As defined herein, anti-hypertensive agents include compounds belonging to a number of therapeutic classes based upon their above-mentioned action and/or mechanism of action, even though the therapeutic outcome is the same, i.e., normalization of circulatory response. It will be appreciated that choice of such anti-hypertensive agents according to the invention is based not solely on the ability of an agent to lower blood pressure; rather, the choice is made with a view to effecting one or more of the above-mentioned actions.

Anti-hypertensive agents suitable for the method of this invention include compounds which inhibit the enzyme which converts the less potent decapeptide vasoconstrictor, angiotensin-I, to the more potent octapeptide vasoconstrictor, angiotensin II (angiotensin-II converting enzyme inhibitors or “ACE inhibitors”), as well as agents which block the binding of angiotensin-II to the AT₁ receptor (“angiotensin-II receptor antagonists”). Anti-hypertensive agents useful in the method of the present invention also include agents which act to decrease intracellular calcium ion concentration in arterial smooth muscle (“calcium channel blockers”), as well as vasodilating agents, such as those which act at α₁-adrenergic receptors or β-adrenergic receptors in the smooth muscle of vascular walls (“α₁-adrenergic receptor antagonists” and “β-adrenergic receptor antagonists”), and endothelin receptor antagonists.

Examples of suitable ACE inhibitors for use in the present invention include benzapine compounds such as benazepril and libenzapril; 6H-pyridazino[1,2-a]diazepine derivatives such as cilazapril; 2,3-dihydro-1H-indene compounds such as delapril; L-proline derivatives such as alacepril, captopril, ceronapril, enalapril, fosinopril, lisinopril, moveltipril and spirapril; oxoimidazoline derivatives such as imidapril; 1,4-dihydropyridine compounds such as lacidipine; iso-quinoline carboxylic acid derivatives such as moexipril and quinapril; 1H-indole carboxylic acid derivatives such as pentopril and perindopril; hexahydroindole carboxylic acid derivatives such as trandolapril; cyclopenta[b]pyrrole carboxylic acid derivatives such as ramipril; and 1,4-thiazepine compounds such as temocapril. Examples of calcium channel blockers include benzothiazepine compounds such as diltiazem; dihydropyridine compounds such as nicardipine, nifedipine, and nimopidine;

phenylalkylamine compounds such as verapamil; diarylaminopropylamine ether compounds such as bepridil; and benimidazole-substituted tetralin compounds such as mibrefadil.

Examples of angiotensin-II receptor antagonists include eprosartan, irbesartan, losartan, and valsartan.

In addition, agents which are not of particular use in treating hypertension, such as lipid-modifying agents (i.e., cholesterol- and triglyceride-lowering agents; for example, anti-hyperlipidemics such as statins, which are HMG CoA reductase inhibitors, and fibric acid derivatives or “fibrates”) are contemplated as useful in the methods of the invention. Further agents include PDE inhibitors, such as PDE-5 inhibitors. It is expected that such agents will also be useful in improving or normalizing sexual response in accordance with the invention.

In embodiments employing chronic anti-hypertensive therapy, the agent is administered in doses regularly used to treat hypertension, or optionally administered in low doses ranging between about one-twentieth to about one-half the dose required to evoke vasodilation in a subject exhibiting normal circulation. In embodiments employing brief anti-hypertensive therapy, the agent is administered over a period of time ranging between about five days to about 21 days at low doses as described above, doses regularly used to treat hypertension, or higher doses ranging between about 1.5 to about 3 times the dose required to evoke vasodilation in a subject exhibiting normal circulation. It will be appreciated that the goal of anti-hypertensive therapy according to the invention is to achieve a normalized, improved, and/or enhanced, and preferably stable circulation, using chronic or brief therapy, or a combination of the two therapies. Brief therapy, as defined herein, has the advantage that it provides periods in which the subject a normalized, improved, and/or enhanced circulation free of any anti-hypertensive molecule. Hence restored central sexual response can be evaluated in the absence of any possible confounding effects of the anti-hypertensive molecule.

As used herein, the terms “centrally-acting dopaminergic sexual response agonist” and “centrally-acting dopaminergic receptor agonist” are intended to include compounds which act upon the mesencephalon or dopaminergic mid-brain pathway to increase blood flow to the ilio-hypogastric-pudendal arterial bed and genitalia, or act upon a mid-brain dopaminergic pathway to stimulate genital vasodilation, engorgement, and lubrication.

Dopaminergic agonists according to the methods of the invention encompass factors that bind to, for example, D1, D2, D3, or D4 receptors, or to more than one receptor category. Examples of dopaminergic pathway compounds are apomorphine, bromocriptine, lisuride, methergoline, pergolide, piribidil, and quinpirole. The preferred centrally-acting compound for use in the methods of the invention is apomorphine, which is known to bind to more than one receptor category, including D1 and D2 categories. For an optimal vasocongestive arousal response in the female, steady-state circulating serum and mid-brain tissue levels of apomorphine should be maintained within a relatively closely defined range. The drug is preferably administered in a formulation which delivers the drug to the system while maintaining and not exceeding the desired systemic levels of the drug. Methods know to the practitioner of the pharmaceutical formulation arts which accomplish this may be used. For example, the drug may be delivered to the system by means of a solid oral formulation, by a liquid formulation, including one applied sub-lingually; by a tablet, lozenge, or lollipop held in the mouth and absorbed buccally; by means of a suppository formulation administered intravaginally or rectally; by a power, gel, or suspension, or an intra-nasal spray formulation. Formulations for the intra-nasal administration of apomorphine are taught, for example, in U.S. Pat. No. 5,756,483 to Merkus; buccal or sub-lingual formulations for the administration of apomorphine are taught in U.S. Pat. No. 5,888,534 to El-Rashidy, et al. See also U.S. Pat. No. 5,770,606 to El-Rashidy, et al., and U.S. Pat. No. 6,395,744 to Adams, et al.

The drug may also be administered in a sterile parenteral formulation by sub-cutaneous or intramuscular route, although sub-lingual, buccal, intra-nasal, and suppository formulations are preferred because of their greater ease of administration and their resulting greater potential for patient acceptance. Sublingual dosage forms, usually containing about 1 mg to about 13 mg, preferably about 1 mg to about 10 mg of apomorphine, more preferably about 2 mg to 10 mg of apomorphine, are useful in treating the symptoms of sexual dysfunction, including its symptomatic manifestations without nausea or other undesirable side effects. Plasma concentrations of apomorphine are preferably at between about 0.1 to 6 nanograms per milliliter, preferably between about 0.3 to about 4 nanograms per milliliter, and more preferably between about 1 to about 2 nanograms per milliliter, sufficient to induce penile erection adequate for intercourse, clitoral erection, vaginal and labialar engorgement and lubrication adequate for intercourse (i.e., effective vasocongestive arousal) but less than the amount that induces nausea.

The apomorphine is administered in the time period immediately prior to sexual activity, generally during the period between about 2 minutes and 120 minutes prior to sexual activity, preferably during the period between about 2 minutes and about 60 minutes prior to sexual activity, so as to achieve desired serum and mid-brain tissue levels of the drug.

Apomorphine has been recognized for use as an emetic when administered subcutaneously in about a 5 milligram dose. For the purposes of the present invention, apomorphine or a similarly acting dopamine receptor agonist is administered in an amount sufficient to excite cells in the mid-brain region of the patient but with minimal side effects. This-cell excitation is believed to be part of a cascade of stimulation that is likely to include neurotransmission with serotonin and oxytocin.

The dopamine receptors in the mid-brain region of a patient can be stimulated to a degree sufficient to cause an erectile response by the administration, preferably sublingually, of apomorphine so as to maintain a plasma concentration of apomorphine of no more than about 5.5 nanograms per milliliter (5.5 ng/ml). The sublingual administration usually takes place over a time period in the range of about 1 to about 10 minutes, or longer. The amount of apomorphine administered sublingually over this time period is preferably in the range of about 10 micrograms per kilogram (μg/kg) of body weight to about 100 μg/kg of body weight, more preferably from about 25 μg/kg to about 80 μg/kg of body weight.

In accordance with another aspect of the invention, there is provided a method of treating female sexual dysfunction comprising administering to a female mammal an anti-hypertensive therapy and a centrally-acting dopaminergic receptor agonist. In one embodiment, the female sexual dysfunction is female sexual arousal disorder (FSAD). In a preferred embodiment, the anti-hypertensive therapy comprises administration of an ACE inhibitor, and the centrally-acting dopaminergic receptor agonist is administered on an as-needed basis. In another embodiment, the anti-hypertensive agent and the centrally-acting dopaminergic receptor agonist are administered on a continuous basis. In a more preferred embodiment, the ACE inhibitor is enalapril, and the dopaminergic receptor agonist is apomorphine.

In accordance with another aspect of the invention, there is provided a method of restoring or enhancing efficacy of a centrally-acting dopaminergic receptor agonist for treating sexual dysfunction, such as FSAD, in a female mammal, comprising administering to a female mammal an anti-hypertensive therapy and a centrally-acting dopaminergic receptor agonist.

In accordance with another aspect of the invention, there is provided a method of treating male sexual dysfunction comprising administering to a male mammal an anti-hypertensive therapy and a centrally-acting dopaminergic receptor agonist. In one embodiment, the male sexual dysfunction is penile erectile dysfunction (ED). In a preferred embodiment, the anti-hypertensive therapy comprises administration of an ACE inhibitor, and the centrally-acting dopaminergic receptor agonist is administered on an as-needed basis. In another embodiment, the anti-hypertensive agent and the centrally-acting dopaminergic receptor agonist are administered on a continuous basis. In a more preferred embodiment, the ACE inhibitor is enalapril, and the dopaminergic receptor agonist is apomorphine.

In accordance with another aspect of the invention, there is provided a method of restoring or enhancing efficacy of a centrally-acting dopaminergic receptor agonist for treating sexual dysfunction, such as ED, in a male mammal, comprising administering to a male mammal an anti-hypertensive therapy and a centrally-acting dopaminergic receptor agonist.

As used herein, the term “sexual dysfunction” is intended to include all aspects of male and female sexual dysfunction and urogenital aging. Male sexual dysfunction includes, but is not limited to, conditions associated with urogenitalia as affected by diabetes, vascular disease, hypertension; sexual arousal disorders; and penile erectile dysfunction.

As used herein, the term “female sexual dysfunction” or “FSD” is intended to include aspects of female dysfunction and urogenital aging such as decreased vaginal lubrication, decreased vaginal engorgement, sexual pain such as, for example, dyspareunia, vaginismus, and vulvodynia; urogenital infections; and urogenitalia as affected by post-menopause, diabetes, vascular disease, hypertension, estrogen depletion conditions, sexual dysfunction, and idiosyncratic vaginal dryness, respectively. It is to be understood that the term “female sexual dysfunction” or “FSD” as used herein encompasses female sexual arousal disorders “FASD” and orgasmic disorders.

The terms “treating” and “amelioration” or “ameliorating” as used throughout this specification and the appended claims are interchangeable and are intended to mean the amelioration, inhibition, or reversal of the symptoms of sexual dysfunction, so as to restore the centrally-initiated sexual response. Restoration of the centrally-initiated sexual response provides for appropriate sexual response to administration of a dopaminergic receptor agonist, such as apomorphine.

According to another aspect of the invention there is provided a single dosage unit of a pharmaceutical composition comprising an anti-hypertensive therapy and a centrally-acting dopaminergic receptor agonist, and a pharmaceutically acceptable carrier therefore. The single dosage unit of the invention is suitable for treating male and female sexual dysfunction, as described herein. In one embodiment, the anti-hypertensive therapy is an ACE-inhibitor and the dopaminergic receptor agonist is apomorphine. In a preferred embodiment, the ACE inhibitor is enalapril, and the dopaminergic receptor agonist is apomorphine.

In a pilot study, described in detail in Example 2, the reduced APO-induced GVA response of aged (18 month-old) female rats, relative to control (young) rats, was treated by brief administration of the anti-hypertensive agent enalapril. Following two weeks of daily anti-hypertensive treatment, APO-induced GVA response of aged rats increased to levels of young rats. The anti-hypertensive treatment also resulted in more of the aged rats responding to the APO stimulus, relative to the number responding pre-treatment. Moreover, the increased sexual responsiveness to APO persisted for at least ten weeks after cessation of treatment, compared to pre-treatment levels. This result indicates that anti-hypertensive treatment produces long term restorative effects in the centrally-initiated sexual response, and that anti-hypertensive treatment is effective if provided periodically.

In a further study, described in detail in Example 3, the age-related loss of apomorphine-induced penile erectile response was treated with enalapril or losartan, resulting in significant increases in the number of erections relative to pre-treatment number of erections.

Pharmaceutical Compositions

Pharmaceutical compositions which are useful in the method of the present invention comprise one or more compounds defined above formulated together with one or more non-toxic pharmaceutically acceptable carriers. The pharmaceutical compositions may be specially formulated for oral administration in solid or liquid form, for parenteral injection, or for vaginal or rectal administration. The formulations may, for example, contain a single therapeutic agent selected from ACE inhibitors, angiotensin-1 (AT₁) receptor antagonists, calcium channel blockers, or a combination of two or more agents selected from the same or different therapeutic categories. Moreover, a combination of one or more therapeutic agents from the groups listed above may be combined with a diuretic agent of the class well known in the art.

To enhance delivery to genital vasculature, combined systemic delivery with topical administration of an erectogenic initiator is also contemplated within the scope of this invention. In this manner the active agent is delivered to target regions at a markedly enhanced rate.

The pharmaceutical compositions of this invention can be administered either systemically or locally to humans and other animals. Systemic routes include oral, parenteral, intracisternal, intraperitoneal, trans-cutaneous (by injection or patch), buccal, sub-lingual administration, or by means of an oral or nasal spray. The term “parenteral” administration as used herein refers to modes of administration which include intravenous, intramuscular, intraperitoneal, intrasternal, subcutaneous and intraarterial injection and infusion. Local administration routes include vaginal, rectal, intraurethral, trans-urethral, by intra-cavernosal injection, or topical administration.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous careers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various and bacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug it is desirable to slow the release or absorption of the drug following subcutaneous or intramuscular rejection. This may be accomplished by the use of a liquid suspension of crystalline or amorphous material with low water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(othoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and 1) lubricants such as talc, calcium stearate, magnesium stearate, solid polyethylene glycols, sodium lauryl sulfate, and mixtures thereof. In the case, of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut com, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Compositions for rectal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods for the formation of liposomes are known in the art. See, for example, Prescott, Ed., Methods in Cell Biology, Volume XIV Academic Press, New York, N.Y. (1976), p. 33 et seq.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated. However, it is well known within the medical art to determine the proper dose for a particular patient by the “dose titration” method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed for the use of the compound as an anti-hypertensive agent. In a chronic, or long-term dosing regimen to remodel the vasculature in the genitalia and in vascular beds feeding the genitalia, lower doses ranging between about 1/20 to about ½ the doses normally given to combat hypertension are used. In short term, acute, or “burst-mode” therapy, the compounds are administered in doses ranging between 1 to 3 times the amounts generally prescribed for hypertension. In these situations, however, appropriate precautions should be taken by the attending physician to closely monitor untoward side-effects peculiar to each particular therapeutic agent.

For the preferred therapeutic agents in the method of the present invention, namely ACE inhibitors, generally dosage levels of about 1 mg to about 300 mg, more preferably of about 5 mg to about 150 mg of active compound per kilogram of body weight per day are administered orally to a patient, with the dose levels appropriately adjusted if the route of administration is other than oral. If desired, the effective daily dose may be divided into multiple doses for purposes of administration, e.g. two or more separate doses per day.

The contents of all cited publications are incorporated herein by reference in their entirety.

The invention is further described by way of the following non-limiting examples.

### WORKING EXAMPLES

### Example 1

### Development of an Animal Model of Female Sexual Function

Animals

Eight adult female Wistar rats (225-250 g) aged 3 to 4 months (Charles River Laboratories, Montreal, Canada) were housed in polypropylene shoebox cages in a climate-controlled room with a 12/12-hour light/dark cycle. Food (Purina® rat chow) and water were provided ad libitum except during times of testing. The procedures carried out in this study were in accordance with the Canadian Council of Animal care. Rats were handled for 5 consecutive days prior to experimentation to allow for acclimation to the investigator. Prior to experimentation, all rats showed a consistent 4-day estrous cycle consisting of normal periods of proestrus, estrus, metestrus and diestrus.

Experimental Procedure.

A modification of a previous protocol using APO (80 ug/kg in 100 ug/ml ascorbic acid in water, s.c.) was used⁽¹⁷⁾. In preliminary studies, a dose-response assessment (APO—40, 80 & 120 ug/kg) demonstrated that a dose of 80 ug/kg APO, elicited the greatest number of genital grooming responses in the female rat. Rats were placed in hanging cages with bottoms constructed of Plexiglas®, in a dark, quiet room where they were allowed to acclimate for 10 minutes before the testing period. Following APO administration, the number of genital responses and associated yawning responses were recorded for a 30 minute period via a video monitoring system in an adjacent room. A genital response was counted when there was a distinct change in the appearance of the external genitalia accompanied by oral grooming of this region. A yawn was counted as an involuntary opening of the mouth with the associated respiratory movement.

Experiment A: APO-Induced Responses in Intact Rats

APO-induced genital responses and yawning responses were determined during each stage of the estrous cycle—proestrus (˜12 h), estrus (˜12 h), metestrus (˜21 h), diestrus (total of ˜57 h)⁽²⁰⁾. Early diestrus was considered to be the 1^{st} half and late diestrus the 2^{nd} half of this segment of the estrous cycle. Stages of the estrous cycle were determined from vaginal smears obtained from each rat^{(19,20,21)}.

Experiment B: APO-Induced Responses in Ovariectomized Rats

APO-induced responses were determined in ovariectomized (OVX)⁽²²⁾ rats to determine the effect of reduced levels of endogenous hormones. In brief, animals were anaesthetized with isoflurane gas delivered via a Bain rebreathing system. A small midline dorsal skin incision was made approximately half-way between the middle of the back and the base of the tail. Both ovaries were removed by ligating and severing the junction between the fallopian tube and uterine hom. Post-operative analgesia (buprenorphine, Buprenex™, 0.05 mg/kg) and antibiotics (0.1 ml/100 g body wt., Tribrissen™ 24%, Schering Canada Inc.) were administered daily for 5 days. Animals were given at least one week to recover before the onset of experimentation.

Responses in Female Wistar Rats to Saline and Apomorphine (APO)

APO (80 ug/kg) administration induced a distinct change in the morphology of the external genitalia of the female rat which was preceded by a patterned behavioral response. A genital vasocongestive arousal (GVA) event was characterized by significant engorgement of the region surrounding the introitus that lasted approximately 3 seconds. The patterned behavioral response involved a ‘startle’ response in which a rat's awareness of its surroundings abruptly increased. This was followed by concavity of the back, then rearing onto hind legs, followed by a rapid head descent toward the engorged genital area, and genital grooming. Genital grooming without any of the other patterned behaviors and vaginal engorgement was scored as a non-GVA event.

APO Administration in Intact Rats

APO was administered to 8 female Wistar rats (250-275 g) in each stage of the estrous cycle and the number of responses was counted in the subsequent 30 minutes. APO administration resulted in a significant increase (Student's t-test p<0.05) in the average number of GVA responses in each stage of the estrous cycle (peaking in estrus), relative to control animals (proestrus: 1.5±1.06 vs. 0.13±0.35; estrus: 1.75±1.67 vs. 0; metestrus: 1.5±1.20 vs. 0.13±0.35; diestrus 1:1.13±1.36 vs. 0; diestrus II: 0.88±0.85 vs. 0.13±0.35). There was no significant difference in non-GVA responses between APO and control tested animals in any stage of the estrous cycle.

Corresponding yawning responses in APO-tested animals was observed in all stages of the estrous cycle in intact animals (2±2.50 in diestrus 1 to 3.63±3.96 in metestrus). There was no significant difference in yawning response among all stages of the estrous cycle. No yawns were observed in control animals.

The distribution of APO-induced GVA responses over the 30-minute period showed GVA responses peaking in the first 5-10 minutes with 80% of total GVA responses occurring within the first 20 minutes. The distribution of APO-induced yawning responses over the 30-minute period showed the yawning responses peaking in the first 15-20 minutes.

APO Administration in Ovariectomized (OVX) Rats

APO-induced GVA responses were determined in 8 OVX Wistar rats. During the 30 minute period, GVA responses were reduced by about 80% in OVX rats relative to intact animals. There was no significant difference between OVX and intact animals in non-GVA responses, in both APO administration and vehicle. APO-induced yawns were observed in both OVX and intact animals.

### Example 2

### Recovery of APO-Induced Sexual Response in Aged Female Rats Using Short-Term Anti-Hypertensive Treatment

Methods

Aged (18 months) and control (3-4 months) female Wistar rats were assessed for APO-induced GVA response as described above (Example 1). Aged females were then treated with enalapril (30 mg kg⁻¹day⁻¹, orally)+low salt diet for 2 weeks. Prior to, during, and after treatment, apomorphine (80 ug kg⁻¹, s.c.) was used to elicit GVA responses. The frequency of GVA responses per rat for a 30-minute period was determined as described above (Example 1).

Results

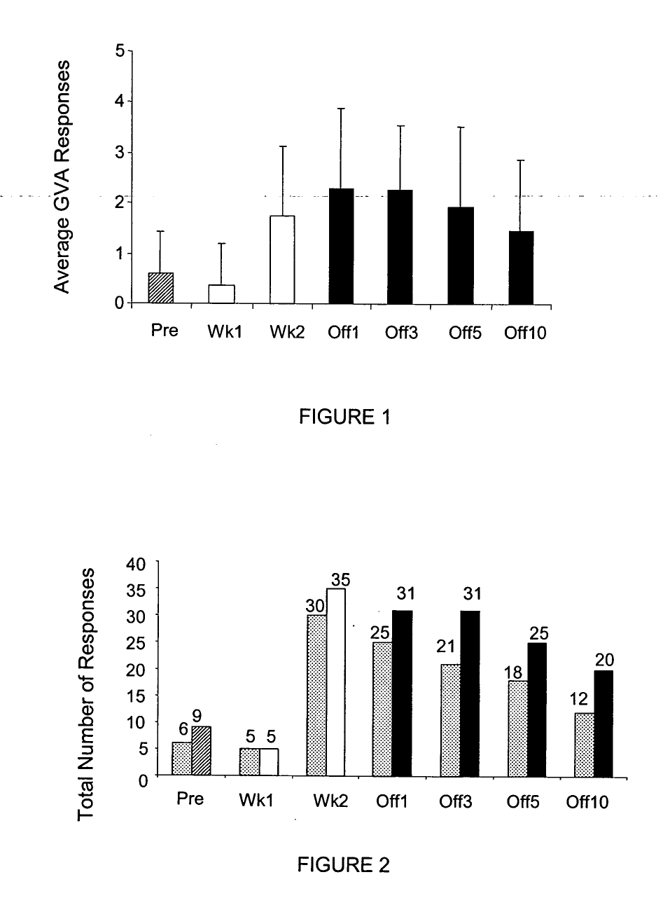

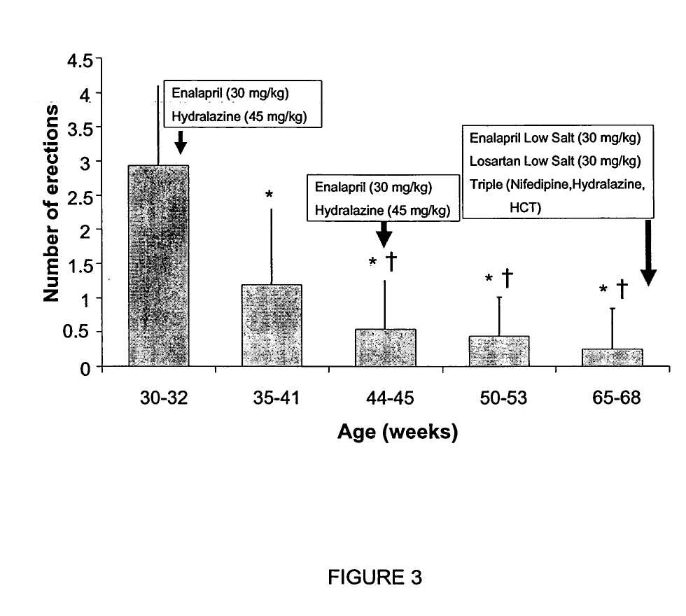

Prior to anti-hypertensive treatment, there were significantly fewer GVA responses in aged female rats relative to controls, which had an average of about 2 GVA responses in the 30 minute period. As shown in FIGS. 1 and 2, during anti-hypertensive treatment of the aged females there was a trend of increasing APO-induced GVA responses (p=0.065). Following cessation of treatment, at 1 & 3 week off-treatment, there was a persistent and significant increase (p<0.05) in frequency of APO-induced GVA responses relative to the treatment period. The average numbers of off-treatment responses at weeks 1 and 3 were similar to those found for young females. FIG. 2 also shows that there was an increase in the number of partial behavioural responses (corresponding hatched bars) that persisted after cessation of the treatment. A partial behavioural response is defined as a genital vasocongestive arousal event without the complete spectrum of behavioural changes, culminating in genital grooming.

In addition, during pre-treatment there was only a 40% APO-induced GVA response rate in aged females. During anti-hypertensive treatment, the overall APO-induced GVA response rate increased to 56% and then further increased significantly to 93% off-treatment. Yawns were observed at similar frequencies in all experimental periods, demonstrating that central activation by APO occurred independently of the genital responses.

### Example 3

### Recovery of APO-Induced Erectile Response in Aged Male Rats Using Short-Term Anti-Hypertensive Treatment

Introduction

Brief, aggressive anti-hypertensive therapy has been shown to recover erectile function in aged spontaneously hypertensive rats (SHR). The present study sought to determine the impact of anti-hypertensive and hormonal treatments on age and hypertension-related changes in apomorphine-induced erections.

Methods

Animals: 30-week-old male spontaneously hypertensive rats (Charles River Laboratories, Montreal, Quebec, Canada) were housed individually at a temperature of 22-24° C. with a 12-hour light/dark cycle. They were provided with free access to regular rodent chow (0.4% Na⁺) and tap water. All procedures were performed in accordance with the guidelines of the Canadian Council on Animal Care.

Anti-hypertensive Treatments. Rats were randomly divided into three groups. The control animals received only tap water. One treatment group received the ACE inhibitor enalapril maleate (30 mg/kg per day), and the other received the vasodilator hydralazine (45 mg/kg per day) (enalapril and hydralazine supplied by Sigma Chemical Co., St. Louis, Mo.) administered orally through their drinking water for 2 weeks. Aspartame (100 mg/1) was added to the hydralazine solution to improve the taste. Drug concentrations were calculated based on previously established drinking patterns⁽²⁴⁾. Several times throughout the 2-week treatment period, the drug concentrations in the drinking water were adjusted to account for fluctuations in animal weight or fluid intake. Erectile response was measured in all groups with the administration of apomorphine (APO). APO-testing was performed three times before treatment and twice between days 7-14 of treatment. Continuous monitoring of erectile response took place until a sustained decrease in erectile function was seen.

A second two-week anti-hypertensive treatment was performed at 49 weeks of age. Rather than trying to prevent erectile dysfunction, this treatment attempted to restore erectile function. The same drugs and procedures were used as outlined above. APO-testing was performed twice between days 7-14 of treatment and twice off treatment.

All rats underwent a third two-week anti-hypertensive treatment at 68 weeks of age to further improve erectile function. Rats were randomized into four new treatment groups. The previously untreated rats received enalapril (30 mg/kg per day) or losartan (30 mg/kg per day) in their drinking water and were fed a low sodium diet (0.04% Na⁺). After 6 days of treatment, a daily 4-hour access to salt containing regular chow (0.4% Na⁺) was permitted to allow for a depressor response of 50% of the pre-treatment levels⁽²⁵⁾. The treated rats received enalapril (30 mg/kg per day) in their drinking water and a low sodium diet (0.04% Na⁺) or a triple therapy treatment⁽²⁴⁾ combining hydralazine (45 mg/kg per day), a calcium channel blocker, nifedipine (200 mg per day) and a diuretic, hydrochlorothiazide (100 mg/l) (all drugs supplied by Sigma Chemical Co., St. Louis, Mo.). Hydralazine and hydrochlorothiazide were administered in the drinking water and nifedipine was mixed into ground regular rodent chow during the 12 h dark cycle. Aspartame (100 mg/l) was added to the hydralazine solution to improve the taste. APO-testing was performed twice between days 7-14 of treatment and twice off treatment.

Testosterone Treatment At 47 and 67 weeks of age, all rats underwent testosterone treatment to determine if the decreased erectile response was due to a hormone deficiency. Testosterone was prepared from a stock solution of testosterone propionate (Taro Pharmaceuticals, Bramalea, Ontario) diluted in peanut oil. Each rat received a single dose of testosterone (480 μg/kg) administered via subcutaneous injection in the neck. Testosterone was administered 36 hours prior to APO-testing. A study by Brien et al.⁽¹⁸⁾ showed erectile recovery to approach the erection average of the controls 36 hours after testosterone administration.

Assessment of Erectile Response. Erectile response was assessed using the well-established bio-assay rat model developed by Heaton et al.⁽¹⁷⁾. The rats were placed in separate, wire hanging-wall cages with clear Plexiglas bottoms in a dark, quiet room and allowed to acclimate for 20 minutes. Each rat received apomorphine (80 μg/kg) in saline (80 μl/ml apomorphine plus 100 μg/ml ascorbic acid) via subcutaneous injection (1 ml/kg) in the loose skin of the neck or back. Erections and yawns were counted at 5-minute intervals over a 30-minute period via videomonitoring in an adjacent room. Erections were classified as a full behavioural erectile response or a partial response. A full erectile behavioural response is the concave arching of the back, pelvic thrusts followed by the emergence of the engorged glans penis and the distal shaft and immediate grooming of the genital area. A partial response incorporates the same erectile response but lacks the grooming behaviour. A yawn is the wide opening of the mouth with the appropriate respiratory movements. APO-testing was performed before, during and after treatment.

Statistical Analysis. Full erectile behavioural responses and partial responses were grouped and the total number of erections was examined. All data are expressed as the proportion of animals responding per test period and as the average number of erections plus or minus standard deviation with significance at p<0.05. The rats acted as their own controls for testosterone administration and pre, on and off treatment values were compared. Paired, two-way Student's t-tests and one-way analysis of variance (ANOVA) with Newman-Keuls post hoc test was used to determine significance between and within treatment groups.

Results

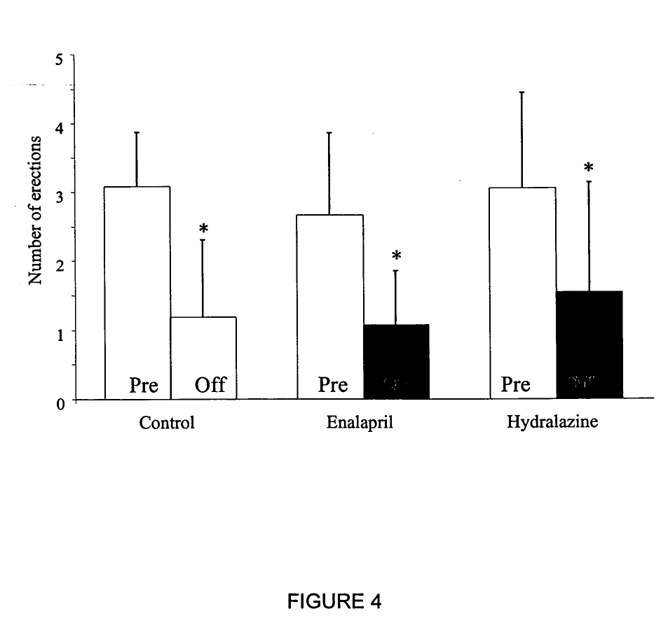

Assessment of APO-induced erectile responses from 30-68 weeks revealed that there was a progressive loss of response in the aging SHR (see FIG. 3). In some of the rats treatments were initiated at 30-32 weeks and again at 44 and 45 weeks. In FIG. 3, asterisks indicate significantly different from 30 weeks, and crosses indicate significantly different from 35 weeks.

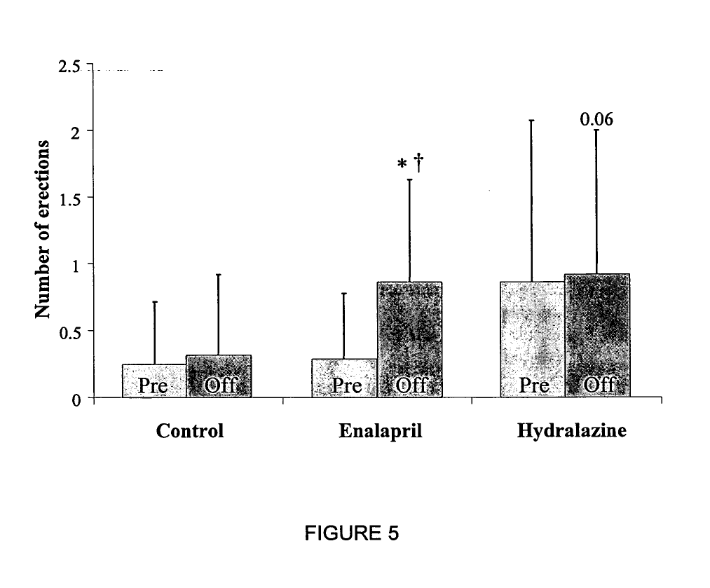

Brief, moderate anti-hypertensive therapy at 30 weeks of age did not prevent or delay the onset of erectile dysfunction (FIG. 4). The average number of erections decreased over time in the control group as previously described. Both moderate treatment groups show the same trend indicating that treatment had little or no prevention effect. Although not shown, yawns were not affected by treatment and remained at an average 12.6±STD throughout all test periods within all three groups.

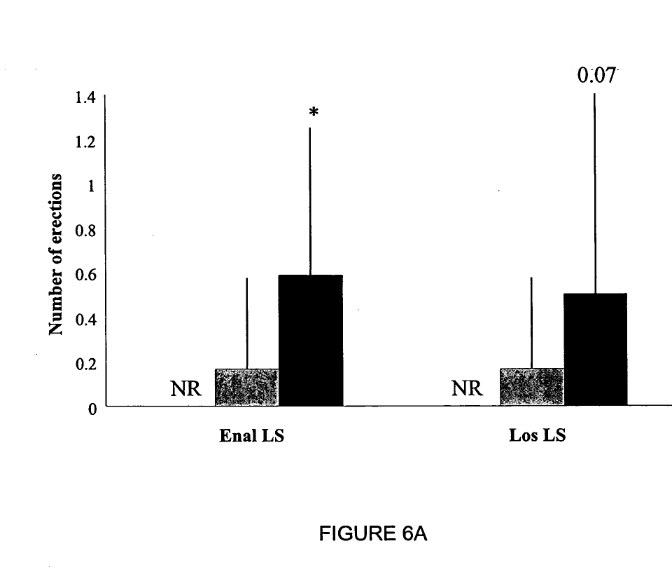

The second anti-hypertensive treatment at 49 weeks showed a trend towards improved erectile function (FIG. 5). The enalapril treated rats had improved sexual function with more than a 2-fold increase in the average number of erections on treatment compared to their pre treatment values although not significant. Two weeks after treatment was withdrawn, the average number of erections increased more and remained at a significantly higher level than the pre treatment values in the enalapril group. The second treatment appeared to have minimal further impact on erectile function in the hydralazine treated rats. Prior to the second treatment the hydralazine rats had a greater number of erections than the enalapril rats and they did not have as great of an improvement off treatment. The proportion of animals responding in both treated groups increased on treatment and remained high after treatment was removed.

Testosterone supplementation at 47 and 66 weeks of age did not significantly affect the number of erections in the control rats and rats treated with hydralazine, although there was an upward trend. The enalapril treated rats had a 2.5-fold increase in the number of erections with testosterone after the first testosterone treatment. However, at 66 weeks of age testosterone did not increase erectile response but did significantly increase the number of yawns.

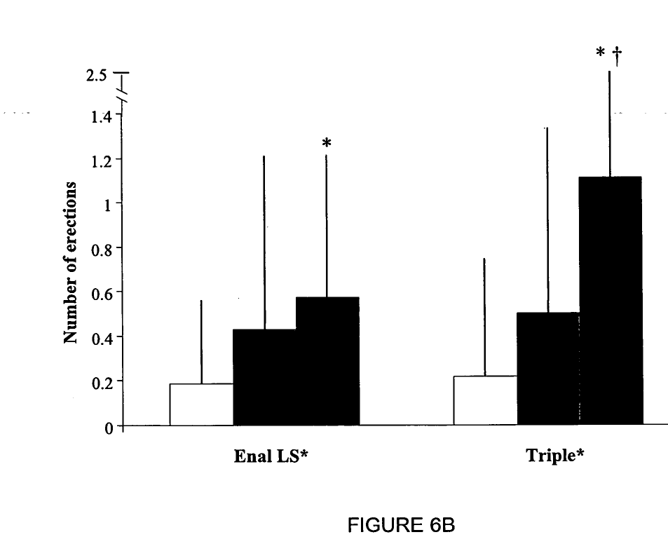

The third anti-hypertensive treatment at 68 weeks of age had a greater impact on the previously untreated rats (FIG. 6A) in comparison to the previously treated ones (FIG. 6B). The first bar of each triplet was at 65 weeks and the middle bar at 68 weeks, 1 week after testosterone administration. The rats treated with enalapril or losartan and a low salt diet had 2-fold increases in the number of erections achieved two weeks off treatment (* is vs first pre-treatment data set). The treated rats that underwent a two-week triple therapy treatment also improved vs both pre-treatment time points

Equivalents

Those skilled in the art will recognize or be able to ascertain, using no more than routine experimentation, variants of the embodiments disclosed herein. Such variants are understood to be within the scope of the invention and are covered by the appended claims.

### Reference List

1. American Psychiatric Association: Diagnostic and statistical manual of mental disorders: DSM-IV, 1994.

2. Laumann, E. O., Paik, A., and Rosen, R. C.: Sexual dysfunction in the United States: prevalence and predictors. JAMA, 281:537, 1999.

3. Berman, J. R., Berman, L., and Goldstein, I.: Female sexual dysfunction: incidence, pathophysiology, evaluation, and treatment options. Urology, 54:385, 1999.

4. Levin, R. J.:VIP, vagina, clitoral and periurethral glans—an update on human female genital arousal. Exp. Clin. Endocrinol., 98:61, 1991.

5. Giuliano, F. and Rampin, O.: Central neural regulation of penile erection. Neurosci. Biobehav. Rev., 24:517, 2000.

6. Goldstein, I. and Berman, J. R.: Vasculogenic female sexual dysfunction: vaginal engorgement and clitoral erectile insufficiency syndromes. Int. J. Impot. Res., 10 Suppl 2:S84, 1998.

7. Ottesen, B., Pedersen, B., Nielsen, J., Dalgaard, D., Wagner, G., and Fahrenkrug, J.: Vasoactive intestinal polypeptide (VIP) provokes vaginal lubrication in normal women. Peptides, 8:797, 1987.

8. Park, K., Goldstein, I., Andry, C., Siroky, M. B., Krane, R. J., and Azadzoi, K. M.: Vasculogenic female sexual dysfunction: the hemodynamic basis for vaginal engorgement insufficiency and clitoral erectile insufficiency. Int. J. Impot. Res., 9:27, 1997.

9. Zemel, P.: Sexual dysfunction in the diabetic patient with hypertension. Am. J. Cardiol., 61:27H, 1988.

10. Kaplan, S. A., Reis, R. B., Kohn, I. J., Ikeguchi, E. F., Laor, E., Te, A. E., and Martins, A. C.: Safety and efficacy of sildenafil in postmenopausal women with sexual dysfunction. Urology, 53:481, 1999.

11. Sodersten, P. and Hansen, S.: Effects of oestradiol and progesterone on the induction and duration of sexual receptivity in cyclic female rats. J. Endocrinol., 74:477, 1977.

12. Foreman, M. M. and Moss, R. L.: Role of hypothalamic dopaminergic receptors in the control of lordosis behavior in the female rat. Physiol Behav., 22:283, 1979.

13. Fernandez-Guasti, A., Ahlenius, S., Hjorth, S., and Larsson, K.: Separation of dopaminergic and serotonergic inhibitory mechanisms in the mediation of estrogen-induced lordosis behaviour in the rat. Pharmacol. Biochem. Behav., 27:93, 1987.

14. La Vaque, T. J. and Rodgers, C. H.: Recovery of mating behavior in the female rat following VMH lesions. Physiol Behav., 14:59, 1975.

15. Maswood, N., Caldarola-Pastuszka, M., and Uphouse, L.: 5-HT3 receptors in the ventromedial nucleus of the hypothalamus and female sexual behavior. Brain Res, 769:13, 1997.

16. Daniels, D. and Flanagan-Cato, L. M.: Functionally-defined compartments of the lordosis neural circuit in the ventromedial hypothalamus in female rats. J. Neurobiol., 45:1, 2000.

17. Heaton, J. P., Varrin, S. J., and Morales, A.: The characterization of a bio-assay of erectile function in a rat model. J. Urol.,1 45:1099, 1991.

18. Brien, S. E., Wilson, E., Heaton, J. P., and Adams, M. A. The conditioned response erection (CRE)—a new approach to modelling erectile responses in the rat. Int. J. Impot. Res., 12:91, 2000.

19. Baker, H. J., Lindsey, J., and Weisbroth, S. H.: The Laboratory Rat. New York, Academic Press 154-155, 1979. 15

20. Hebel, R. and Stromberg, M.: Anatomy and embrylology of the laboratory rat. Gunzberg, Germany, Appel-Druck Donau-Verlag Gmbh. 231-231, 1986.

21. Jaber, L.: Stage specific expression of platelet derived growth factor-AB and its receptors in the mouse uterus during the estrous cycle and early pregnancy. 1995.

22. Waynforth, H. and Flecknell, P.: Specific surgical operations. Experimental and Surgical Techniques in the Rat. London, Academic Press Ltd. 276-277, 1992.

23. Hale, T. M. et al. Recovery of erectile function after brief aggressive anti-hypertensive therapy. J. Urol. 168:348-354, 2002.

24. Woolard, J., Hale, T. M., Bushfield, T. L., Adams, M. A. Persistent lowering of arterial pressure after continuous and intermittent therapy. J. Hypertens. 21:813-820, 2003.

25. Hale, T. M., et al. Time course of vascular structural changes during and after short-term antihypertensive treatment. Hypertension 42:171-176, 2003.

We claim:

1. A method of normalizing sexual response in a subject, comprising administering to a subject in need thereof an anti-hypertensive therapy and a centrally-acting dopaminergic agonist, such that sexual response is normalized.

2. The method of claim 1, wherein the subject is a human male.

3. The method of claim 1, wherein the subject is a human female.

4. A method of ameliorating female sexual dysfunction in a subject comprising administering to a female subject in need thereof an anti-hypertensive therapy and a centrally-acting dopaminergic agonist, such that female sexual dysfunction is ameliorated.

5. The method of claim 4, wherein the female sexual dysfunction is female sexual arousal disorder.

6. A method of ameliorating male sexual dysfunction in a subject comprising administering to a male subject in need thereof an anti-hypertensive therapy and a centrally-acting dopaminergic agonist, such that male sexual dysfunction is ameliorated.

7. The method of claim 6, wherein the male sexual dysfunction is penile erectile dysfunction.

8. A method of enhancing the efficacy of a centrally-acting dopaminergic receptor agonist in the treatment of sexual dysfunction, comprising administering to a subject in need thereof an anti-hypertensive therapy and centrally-acting dopaminergic receptor agonist.

9. The method of claim 1, wherein said anti-hypertensive therapy comprises administration of an ACE inhibitor over a period of time ranging between about five days to about 21 days at a dose ranging between about 1 mg to about 300 mg per kg of body weight per day.

10. The method of claim 9, wherein said ACE inhibitor is enalapril, and said enalapril is administered at a dose of about 30 mg per kg of body weight per day.

11. The method of claim 9, wherein said dopaminergic receptor agonist is apomorphine or a pharmaceutically acceptable salt or ester thereof, administered on an as-needed basis.

12. The method of claim 11, wherein said apomorphine is administered in an amount between about 25 μg/kg of body weight and about 80 μg/kg of body weight.

13. The method of claim 11, wherein the plasma concentration of apomorphine is maintained at a level no more than about 5.5 ng/ml.

14. The method of claim 11, wherein said apomorphine is administered via a route selected from subcutaneous, intramuscular, transdermal, sublingual, buccal, intranasal, vaginal, and rectal.

15. The method of claim 1, wherein the anti-hypertensive therapy and the centrally-acting dopaminergic agonist are administered at the same time.

16. The method of claim 15, wherein the anti-hypertensive therapy and the centrally-acting dopaminergic antagonist are administered together as a single dosage unit.

17. The method of claim 16, wherein the anti-hypertensive therapy is an ACE inhibitor and the centrally-acting dopaminergic antagonist is apomorphine or a pharmaceutically acceptable salt or ester thereof.

18. A pharmaceutical composition comprising a single dosage unit of an anti-hypertensive therapy and a centrally-acting dopaminergic antagonist together with a pharmaceutically acceptable carrier therefore.

19. The pharmaceutical composition of claim 19, wherein the anti-hypertensive therapy is selected from the group consisting of ACE inhibitors, angiotensin II receptor antagonists, calcium channel blockers, α₁-adrenergic receptor antagonists, β-adrenergic receptor antagonists, and endothelin receptor antagonists, and the centrally-acting dopaminergic antagonist is apomorphine or a pharmaceutically acceptable salt or ester thereof.

20. The composition of claim 19, wherein the anti-hypertensive therapy is an ACE inhibitor.
